(19) United States
(12) Patent Application Publication
(10) Pub. No.: US 2014/0148637 A1
(43) Pub. Date: May 29, 2014

(54) Therapeutic Apparatus

(71) Applicants: Asher Gratt, London (GB); Robin Maytum, Luton (GB); Itay Sherman, Hod Hasharon (IL); Jan Domin, Luton (GB)

(72) Inventors: Asher Gratt, London (GB); Robin Maytum, Luton (GB); Itay Sherman, Hod Hasharon (IL); Jan Domin, Luton (GB)

(21) Appl. No.: 14/112,498

(22) PCT Filed: Apr. 10, 2012
(86) PCT No.: PCT/GB12/00318

§ 371 Date: Jan. 3, 2014

(30) Foreign Application Priority Data
Apr. 18, 2011 (GB) 1106527.3

(51) Int. Cl. A61N 2/00

(52) U.S. Cl. CPC A61N 2/002
USPC 600/9

(57) ABSTRACT

The invention relates to a therapeutic apparatus (1) for irradiating biological cells. The apparatus (1) comprising a near field generator (12) comprising an antenna (4) and a signal generator (2). The near field generator (12) being configured to generate a near field signal.

### FIELD

The present invention relates to therapeutic apparatus.

### BACKGROUND

It is known to provide apparatus for influencing the function of biological cells such as cancerous cell growth or proliferation thereof. Such apparatus may include irradiating the biological cells with a microwave field or an electric field. When using a microwave field localised heating of the biological cells is produced, which causes tumour cells to die. Such thermal techniques of this kind have been used successfully to treat cancer.

When using the electric field technique to treat cells, two electrodes are placed on either side of the biological cells to subject them to the electric field. The electrodes are in direct contact with the biological cells and as such the technique may be termed a conduction or contact technique. The electric field used is typically a low intensity electric field in the range 1-2 V·cm⁻¹ which has a fixed frequency in the range 100-300 kHz. Such electric field techniques are still in the early stages of development and have been used successfully to treat cancer cells in clinical trials. Using an electric field is thought to influence the biological cells due to a distortion of microtubule assembly, which is necessary for cell division.

There are many problems associated with the previously known apparatus for treating biological cells. Using microwaves to heat and destroy biological cells may cause unwanted damage, such as heat damage, to surrounding cells. Using electric fields requires electrodes to be placed in contact with the biological cells to be treated, which may be impractical or difficult to achieve. If the electrodes are placed on the skin any hair on the skin is required to be removed by shaving, which may be undesirable. Furthermore, it may not always be practical to use the electric field technique for a prolonged period of time.

It is broadly an object of the present invention to address one or more of the above mentioned disadvantages of the previously known therapy apparatus.

### SUMMARY

What is required is an apparatus and a method of operation thereof which may reduce or minimise at least some of the above-mentioned problems.

In accordance with a first aspect of the present invention, there is provided a therapeutic apparatus for irradiating biological cells comprising a near field generator comprising an antenna and a signal generator, the near field generator being configured to generate a near field signal.

It has been found that an unexpected and surprising effect of such a near field signal is to kill or at least reduce the growth or cell division of biological cells, for example cancerous cells. It will be appreciated that the near field signal is substantially comprised of a magnetic field. Such an effect on the biological cells is due to the magnetic field. Such an apparatus may provide similar or improved results to the prior art electric field conduction techniques mentioned above. According to the present invention the magnetic field generated represents an entirely different approach for treating biological cells. A further advantage is achieved because the apparatus provides a way of irradiating the biological cells in a non-contact manner. The magnetic field used is of a field strength that may also avoid or reduce heating of the biological cells, which may avoid or reduce damage to the surrounding cells due to thermal effects. The near field signal is not modulated by an information signal or data signal.

Preferably the near field generator is configured to modulate the near field signal by a predetermined modulating signal. Such a near field signal may produce a pulsed or varying magnetic field which may be associated with an improved effect on the biological cells.

Preferably the near field signal comprises a carrier signal having a frequency in the range 10 kHz to 20 MHz, the carrier signal being modulated by the modulating signal having a frequency in the range 0.01 Hz to 10 kHz. Such parameters for the carrier signal and the modulating signal are within ranges where the magnetic field may have an effect on the biological cells.

Preferably the modulating signal has a frequency in the range 30-100 Hz. In one embodiment the modulating signal has a frequency in the range 30-60 Hz. Such ranges for the modulating signal may be associated with an effect on the biological cells.

Preferably the carrier signal has a frequency in the range 125 kHz-20 MHz. Preferably the carrier signal has a frequency in the range 11-15 MHz. In one embodiment the carrier signal has a frequency of about 13.56 MHz. Such a ranges for the carrier signal may be associated with an effect on the biological cells.

In one embodiment the modulating signal is a square wave. In one embodiment the modulating signal has a duty cycle of 20 to 80%.

Preferably the modulating signal varies in frequency and/or duty cycle. Preferably the modulating signal varies in frequency and/or duty cycle periodically. Varying the modulating signal in such a manner smears the frequency of the near field signal to reduce regular periodicity of frequencies in the magnetic field. It will be appreciated that varying the modulating signal in such a manner reduces periodicity of the near field signal due to the modulating signal and provides the near field signal with an envelope having a distributed energy.

Preferably the frequency and/or the duty cycle of the modulating signal varies with a period of in the range 0.01 to 1 second. In one embodiment the frequency and/or duty cycle of the modulating signal varies with a period in the range 100-150 milliseconds. Such ranges for the period of the modulating signal may be associated with an effect on the biological cells.

Preferably the near field signal has a field strength of 1 micro Tesla to 100 microTesla at a range of 4 cm or less. In one embodiment the field strength is 15 microTesla at 4 cm or less.

The apparatus may further comprise an RF signal generator configured to generate a periodic RF signal for irradiating the cells in addition to the near field signal. The RF signal may be a periodic communications frequency signal.

It has been found that the effect of a near field signal on its own or in combination with an RF signal will under specific exposure conditions kill, or at least reduce the growth or cell division, of micro-organisms or cells, for example cancerous cells.

In one embodiment the antenna of the near field generator is a directional antenna. The apparatus may comprise an antenna arrangement having a controllable radiation pattern and means for controlling the radiation pattern.

According to a second aspect of the invention there is provided a method of treating biological cells comprising irradiating the cells with a near field signal.

It has been found that such a method provides an unexpected and surprising effect of killing or at least reducing the growth or cell division of biological cells, for example cancerous cells. Such a method may provide similar or improved results to the prior art electric field conduction techniques mentioned above. According to the present invention the magnetic field generated represents an entirely different approach for treating biological cells. A further advantage is achieved because the method provides a way of irradiating the biological cells in a non-contact manner. Such a method of using a magnetic field may also avoid or reduce heating of the biological cells, which may avoid or reduce damage to the surrounding cells due to thermal effects.

Preferably the method further includes modulating the near field signal with a predetermined modulating signal. Such a near field signal may produce a pulsed or varying magnetic field which may be associated with an improved effect on the biological cells.

Preferably the method further including using a near field signal comprises a carrier signal having a frequency in the range 10 kHz to 20 MHz which is modulated by the modulating signal having a frequency in the range 0.01 Hz to 10 kHz. Such a method of using the parameters for the carrier signal and the modulating signal are within ranges where the magnetic field may have an effect on the biological cells.

Preferably the method further includes using a frequency in the range 30-100 Hz for the modulating signal. In one embodiment the method further includes using a frequency in the range 30-60 Hz for the modulating signal. Such a method may be associated with an effect on the biological cells.

Preferably the method further includes using a frequency in the range 125 kHz-15 MHz for the carrier signal. Preferably method further includes using a frequency in the range 11-15 MHz for the carrier signal. In one embodiment the method further includes using a frequency of about 13.56 MHz for the carrier signal. Such a method may be associated with an effect on the biological cells.

In one embodiment the method further includes using a square wave for the modulating signal. In one embodiment the method further includes using a duty cycle of 20 to 80% for the modulating signal.

Preferably the method further includes varying a frequency and/or a duty cycle of the modulating signal. Preferably the method further includes varying the frequency and/or the duty cycle of the modulating signal periodically. Varying the modulating signal in such a manner smears the frequency of the near field signal to reduce regular periodicity of frequencies in the magnetic field.

Preferably the method further includes using a period of the frequency and/or the duty cycle of the modulating signal which varies in the range 0.01 to 1 second. In one embodiment the method further includes using a period of the frequency and/or the duty cycle of the modulating signal which varies in the range 100-150 milliseconds. Such a method may be associated with an effect on the biological cells.

Preferably the method further includes using a field strength of 1 microTesla to 100 microTesla at a range of 4 cm or less for the near field signal. In one embodiment the method further includes using the field strength of 15 microTesla at 4 cm or less.

The method may further include irradiating the cells with a periodic RF signal in addition to the near field signal. The method further includes using a periodic communications frequency signal for the RF signal.

It has been found that the effect of a near field signal on its own or in combination with an RF signal will under specific exposure conditions kill, or at least reduce the growth or cell division, of micro-organisms or cells, for example cancerous cells.

In one embodiment the method further includes controlling a radiation pattern of the near field signal using the directional antenna. The method further includes controlling a radiation pattern of the near field signal using the directional antenna.

According to an alternative characterisation of the invention there is provided a therapeutic apparatus for irradiating biological cells comprising a near field generator comprising an antenna and a signal generator, the near field generator being configured to generate a near field signal, wherein the near field signal substantially comprises a magnetic field.

According to another aspect of the invention there is provided a method of operating the apparatus of the first aspect of the invention using the method of the second aspect of the invention.

Any preferred or optional features of one aspect or characterisation of the invention may be preferred or optional feature of other aspects or characterisations of the invention.

Further features and advantages of the invention will become apparent from the following description of illustrative embodiments of the invention, given by way of example only, which is made with reference to the accompanying drawings.

### BRIEF DESCRIPTION OF THE DRAWINGS

FIG. 1 is a schematic block diagram of an example of apparatus in accordance with the invention;

FIG. 2 is a schematic diagram of an example of a near field signal generator of FIG. 1;

FIG. 3 shows signal diagrams illustrating examples of a near field signal;

FIG. 4 is a signal diagram of an example of periodic RF signal produced by an RF generator of FIG. 1;

FIG. 5 shows a method according to an embodiment of the invention; and

FIG. 6 shows a series of graphs illustrating experimental results.

### DETAILED DESCRIPTION OF ILLUSTRATIVE EMBODIMENTS OF THE INVENTION

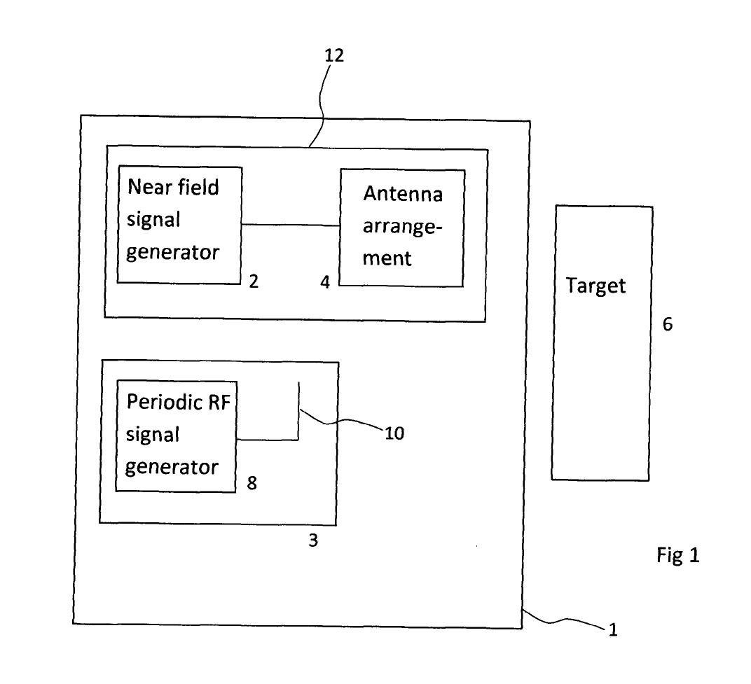

Referring to FIG. 1, an apparatus 1 in accordance with an example of the invention comprises a near field signal generator 2 having an antenna 4. The near field signal generator 2 and the antenna 4 comprise a near field generator 12. The near field generator 12 is a near field signal device which operates by magnetic induction such that an electrical current, i.e. an electric signal, in the antenna 4 produces a magnetic field emitted from the antenna 4 which comprises the near field signal. The antenna arrangement 4 is, for example, an inductor such as a coil, which is capable of producing the magnetic field, and the electrical signal produces an alternating magnetic field from the antenna arrangement 4. It will be appreciated that such a near field generator 12 produces an electromagnetic field, and it is the magnetic component of the electromagnetic field which is the most significant component of the electromagnetic field. The apparatus 1 of FIG. 1 is positioned to irradiate a target 6 with a near field signal, which in one example is modulated by a predetermined modulating signal.

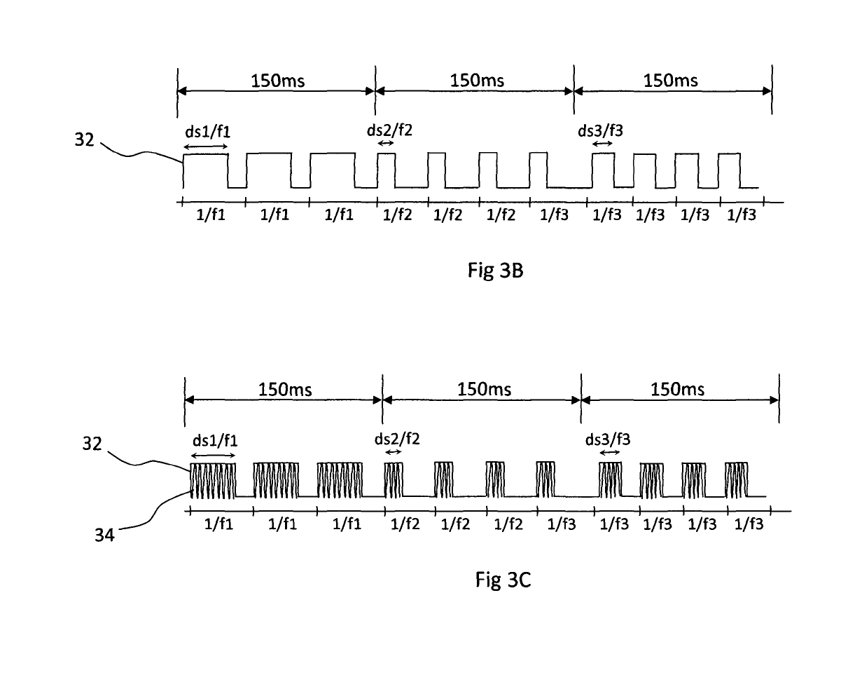

In this example, the target 6 is positioned about 4 to 30 cm, e.g. 10 cm or 20 cm, from the antenna 4. The near field signal generator 2 generates the electrical signal, which produces a near field signal, an example of which is described with reference to FIG. 3. In another example, the target 6 is positioned up to 4 cm from the antenna 4, and in another embodiment the target 6 is positioned about 2-10 mm from the antenna 4. In another embodiment the target 6 is positioned about 2 cm from the antenna 4.

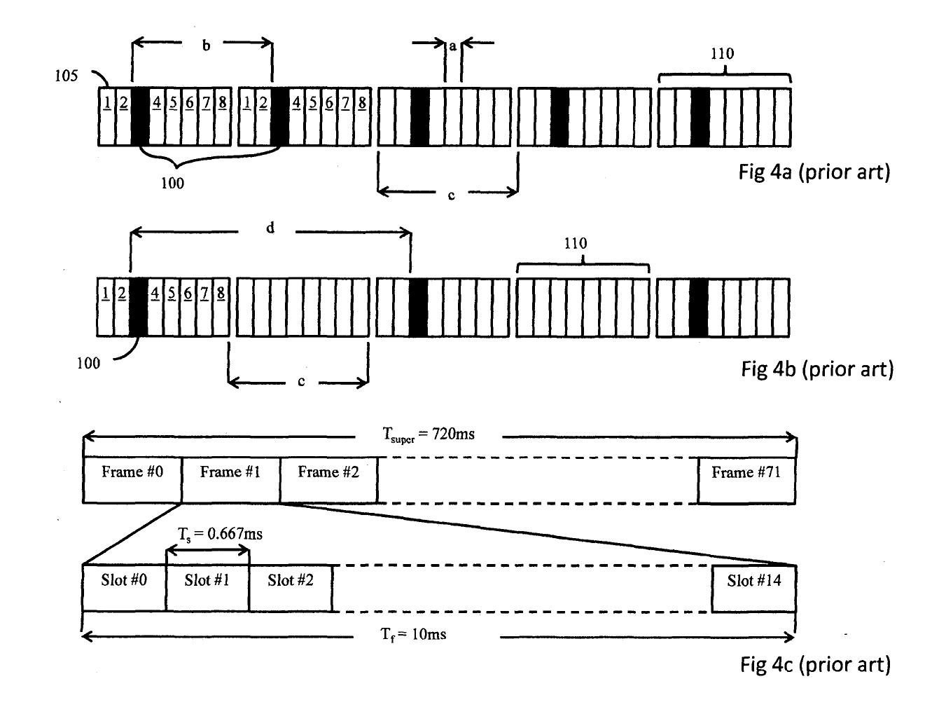

The apparatus 1 may comprise the near field signal generator 2 and antenna 4 alone but in the example shown additionally comprises a Radio Frequency (RF) generator 3 comprising an RF signal generator 8 and its RF antenna 10. The RF signal generator 8 generates a periodic RF signal an example of which is described with reference to FIG. 4. The periodic RF signal is an electromagnetic radio wave.

In FIG. 1, the target 6 may be biological cells, and the apparatus 1 is for the treatment of the cells. It has been found that the effect of a near field signal emitted from the antenna arrangement 4 on its own, or in combination with an RF signal emitted from the antenna 10, will under specific exposure conditions kill, or at least reduce the growth or cell division, of cells, for example cancerous cells. It will be appreciated that the RF signal and the near field signal are emitted or emanated from the RF antenna 10 and the antenna arrangement 4 respectively, and that there is no direct contact such as direct electrical coupling, i.e. conduction of the RF antenna 10 or the antenna arrangement 4, to the biological cells. The embodiments discussed herein are based on inductance and/or radiation of the emitted field, and there is no direct conductance or contact with the target 6. Accordingly, in this context the term irradiating may be considered to include the RF signal emitted from the antenna 10 and the near field signal emitted from the antenna 4.

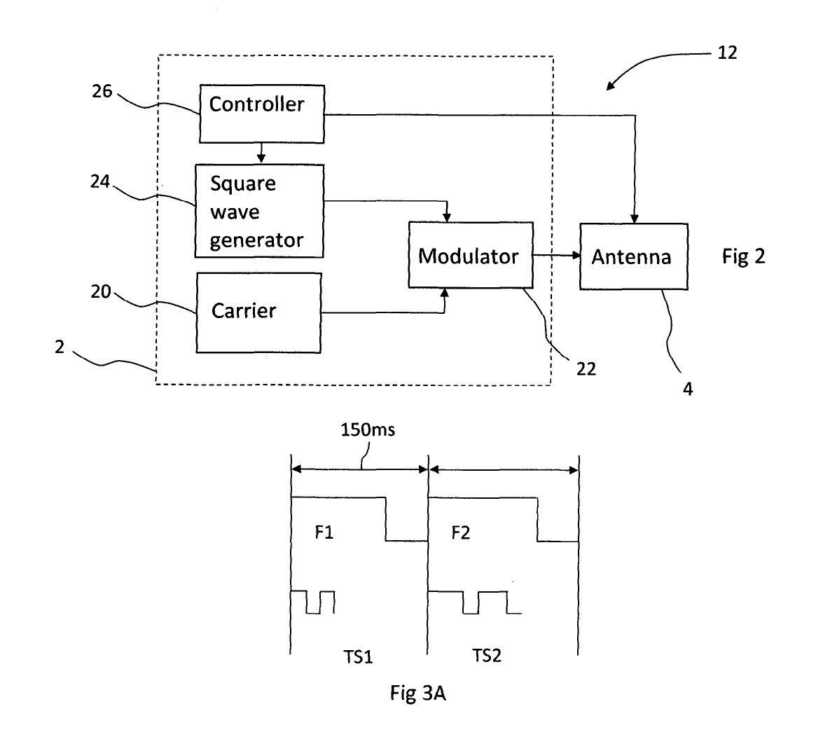

FIG. 2 is an example of the near field generator 12 for producing a near field signal as shown in FIG. 3. In FIG. 2 like features to the arrangements of FIG. 1 are shown with like reference numerals. In FIG. 2 the near field signal generator 2 is shown with a dashed box. The near field signal generator 2 of FIG. 2 comprises a carrier signal generator 20 which produces an RF signal with a frequency in the range 10 kHz to 20 MHz. In another example the carrier signal has a frequency in the range 11 to 15 MHz. In another example the carrier signal has a frequency in the range 125 kHz to 20 MHz. In this example the carrier signal has a frequency of 13.56 MHz. In another example it has a frequency of 125 kHz. The lower bound of 10 kHz and the upper bound 20 MHz for the carrier signal may be defined by the physical limitations of producing an inductor that will operate above 20 MHz and/or the availability of a suitable inductor. Such parameters for the carrier signal are within ranges where the magnetic field may have an effect on the biological cells. The near field signal generator 2 also comprises a modulator 22. The carrier signal is supplied to the modulator 22 where it is modulated by a predetermined modulation signal. The modulating signal does not convey any information or data.

In this example the modulating signal is a square wave. The square wave preferably varies in frequency with time, for example with a period in the range 0.01 to 1 second, and in another example with a period in the range 100 to 150 milliseconds. Accordingly the square wave is a Frequency Modulated (FM) signal. In the example of FIGS. 2 and 3, the square wave is generated by a generator 24 controlled by a controller 26. The near field signal generator 2 also comprises the generator 24 and the controller 26. The frequency of the modulating signal is in the range 0.01 Hz to 10 kHz. In this example the modulating signal has a frequency around 50 Hz. In another example it has a frequency around 8 kHz. In another example the modulating signal is in the range 30 to 100 Hz. In another example the modulating signal has a frequency in the range 30-60 Hz. Such parameters for the modulating signal are within ranges where the magnetic field may have an effect on the biological cells. It will be appreciated that the frequency of the modulating signal should be at a frequency which is lower than the frequency of the carrier signal.

It will be appreciated that when the near field signal comprises a carrier wave which is modulated by a modulating signal that has a changing period, the magnetic field produced from the antenna 4 is pseudo-random within predefined parameters. That is, the magnetic field may vary continually and may not be a fixed single frequency or amplitude for any prolonged period. When a Fourier Transform (FT) is performed on the modulating signal which has a variable frequency, there are no spectral peaks shown in the frequency spectrum. Accordingly the FT is intended to be substantially uniform in the frequency plane. The carrier wave being modulated by a modulating signal that has a changing period aims to smear the frequency of the near field signal to avoid or reduce a regular periodicity of frequencies in the magnetic field. The magnetic field produced has an envelope energy that is distributed across the low frequency range with no or minimal significant peaks. It will be appreciated that the near field signal is a continuously changing magnetic field. The aim of such a magnetic field is to excite the biological cells in a particular manner to kill or at least reduce the growth or cell division of biological cells. In the examples provided herein the levels of energy used for the magnetic field are low, and avoid significant heating of the biological cells.

The upper bound of 10 kHz, and the lower bound of 0.01 Hz for the modulating signal is the maximum and minimum modulating frequency at which the magnetic field has an effect on the biological cells. The upper bound of 1 second, and the lower bound of 0.01 second for the varying period of the modulating signal is the maximum and minimum periods at which the magnetic field has an effect on the biological cells. It will be appreciated that the period of the changing modulating signal should be at a frequency which is lower than the frequency of the modulating signal itself.

As shown in the example of FIG. 3A the square wave is varied in frequency by the controller 26 in time slots TS1, TS2. In a first time slot TS1 it has a first frequency F1 and a second frequency F2 in a second time slot TS2. The time slots TS1, TS2 have a preset duration, for example 150 ms. The controller 26 may control the frequencies in each time slot TS1, TS2 so that the square wave is switched off for part of the time slot e.g. with a duty cycle of 10 to 90%, for example 20 to 80%. The square wave 30, when present in a time slot, itself has a duty cycle of 10 to 90% for example 20 to 80%.

FIG. 3B shows an example of the modulating signal 32 used to modulate the carrier signal. At each 150 ms interval a selected frequency and duty cycle is used for the square wave modulating signal 32. The period is shown as 1/f, and in this example 1/f1>1/f2>1/f3. The duty cycle in each 150 ms interval is shown as ds/f, where ds is the duty cycle expressed as a fraction ranging from 0 to 1, and in this example ds1/f1>ds3/f3>ds2/f2. FIG. 3C shows the modulating signal 32 shown in FIG. 3B combined with the carrier signal 34. The carrier signal 34 is shown to be within the envelope of the modulating signal 32.

The near field generator 12 may produce a near field signal of 1 microTesla to 100 microTesla at a range of 4 cm or less from the antenna 4. In this example the signal field strength i.e. magnetic flux density is 15 microTesla at 4 cm from the antenna 4. The range 1 microTesla to 100 microTesla is a low intensity magnetic field which aims to treat the biological cells whilst reducing or eliminating heating thereof.

The antenna 4 is preferably a directional antenna to direct the signal at the target. The antenna 4 may be controllable, e.g. by the controller 26 to produce a controllable radiation pattern to direct the near field signal to a particular location. The particular pattern and the distance over which the near field signal is effective depends to a large extent on the design of the inductor itself. Such inductors may comprise a coil designed to have a focal point, which may be localised or spread over a relative short range. The focal point is the region at which the biological cells may be effectively treated.

An example of a suitable near field generator 12 that may be used with the embodiments described herein to produce the near field signal may be purchased from Advanced Card Systems Ltd, model ACR122U, which operates at 13.56 MHz and is compliant with the ISO/IEC 18092 Near Field Communication (NFC) standard. Such a near field generator 12 is generally used for near field communications using the NFC standard. According to the embodiments herein such a near field generator 12 is not used to convey any communications data, and is operated to emit a carrier wave at 13.56 MHz which is frequency modulated by a square wave between 30 to 60 Hz. Such a near field generator 12 is connected to a Personal Computer to control it so that it emits the required magnetic field, which is the near field signal. Accordingly, the near field signal comprises the carrier wave of 13.56 MHz, which is a radio frequency and which is frequency modulated by the square wave between 30 to 60 Hz.

The RF generator 3 if provided is periodic. It may produce a signal having a carrier frequency in the range 100 MHz to 6 GHz or higher used for communications, e.g. 300 MHz or above as used for GSM and 3G personal communication devices (PCDs). The RF generator 3 produces a carrier which is periodically pulsed at a frequency lower than the carrier frequency. For example a signal according to the GSM standard or to 3G standard may be generated. It will be appreciated that in the examples of the present invention, no information, whether voice, data or other information, is conveyed by the RF signal applied to the target. The signal power may be 0.5 Watts for example.

All references to GSM herein relate to ETSI TS 144 018 Digital cellular telecommunications system (Phase 2+); Mobile radio interface layer 3 specification; Radio Resource Control (RRC) protocol (3GPP TS 44.018 version 9.4.0 Release 9) unless otherwise specified. Taking the GSM mobile communications standard as an example of the RF signal emitted from the RF generator 3, with reference to FIG. 4a, a communications channel transmits traffic bursts 100 in timeslots 105 that are a=3/52000 s (that is, about 577 μs) long. The bursts 100 may be assigned to timeslots 105 on a one-per-frame 110 basis, where each frame 110 accommodates eight timeslots. In this example, each burst 100 occurs in timeslot 3 of each consecutive frame 110, shown with the timeslot 3 blacked out, to produce a pattern of traffic bursts 100, which is periodic over consecutive timeframes. In effect, the period between bursts is fixed. Accordingly, if the channel is communicated by traffic bursts in one timeslot in each frame, the timeslots occur every b=4.615 ms, which is the same as the frame duration, c. This means that a burst of energy arises every 4.615 ms, which is equivalent to a frequency of 217 Hz. Even if, as illustrated in FIG. 4b, timeslots 100 arise in only every other timeframe, which is the case in half rate GSM, the field generated by the timeslot bursts is at a frequency of 108.5 Hz (that is, pulse spacing d=9.23 ms). It will be appreciated that the illustrations in FIGS. 4a and 4b are merely representative and are not intended to be accurate depictions of GSM.

Taking 3G as another example of the RF signal emitted from the RF generator 3, with reference to FIG. 4c, it can be seen that the signal format has a hierarchical format in which consecutive 720 ms Superframes each comprise 72 Frames, each being 10 ms in duration. Each frame comprises 15 slots, which are 0.667 ms in duration, and each slot includes a power control signal, such that power control signals pulse every 0.667 ms, which corresponds to a frequency of 1.5 kHz. In addition, although not so pronounced as the power control pulses, it has been appreciated also that inter-frame pulsing can occur at 100 Hz in 3G systems.

While GSM type signals may be used, other signals based on GSM, or on any other wireless communication protocol that uses a TDM/TDMA approach in an air interface; such as 2G mobile phone technologies generally (including GSM and others), DECT, Bluetooth, and the like may be used.

It will also be appreciated that, in addition to CDMA, other signal types may be used based for example on frequency division multiple access (FDMA), space division multiple access (SDMA), polarisation division multiple access (PDMA), frequency division duplex (FDD), time division duplex (TDD) and pulse address multiple access (PAMA), to the extent that they generate pulsed RF fields, i.e. periodic RF fields, emitted from the RF generator 3.

The apparatus of the invention may be used to irradiate biological cells for example cancerous cells. Experiments carried out by the applicants have surprisingly shown a biological effect on cell cultures which points towards an apototic effect (cell death) occurring when the near field signal was applied to a culture on its own. The effect was unexpected and was also found when a combination of the near field signal and the RF signal was applied to a culture.

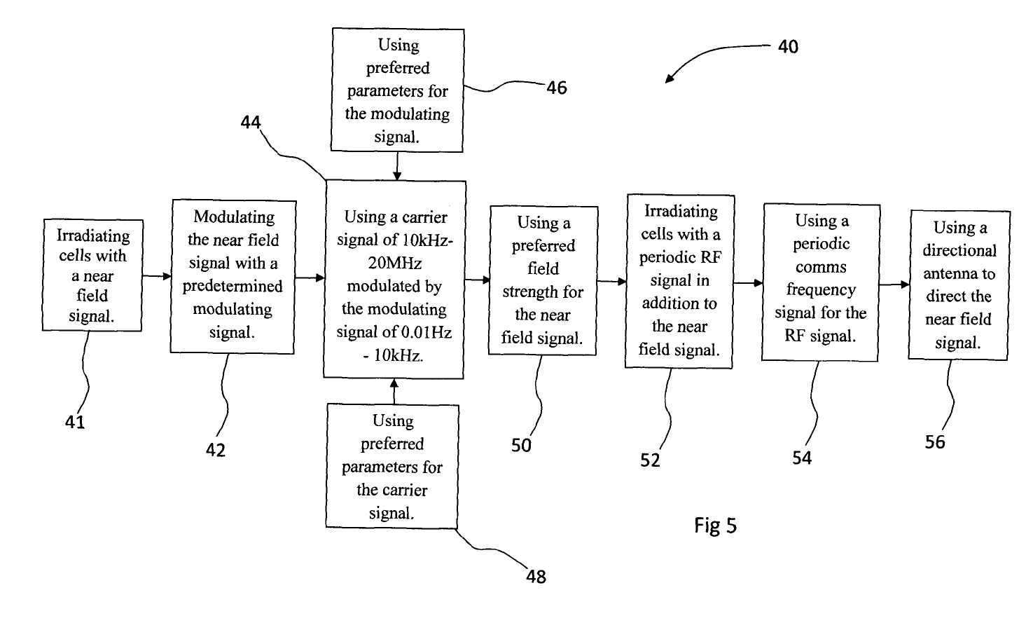

FIG. 5 shows a method according to an embodiment of the invention, generally designated 40. The method 40 is a method of treating biological cells using a therapeutic apparatus 1 for irradiating the biological cells. The therapeutic apparatus 1 comprises a near field generator 12 comprising an antenna 4 and a near field signal generator 2. The method includes irradiating the cells with a near field signal, as shown at 41.

The method further includes modulating the near field signal with a predetermined modulating signal, as shown at 42. The method further includes using a near field signal comprises a carrier signal having a frequency in the range 10 kHz to 20 MHz which is modulated by a modulating signal having a frequency in the range 0.01 Hz to 10 kHz, as shown at 44. The method includes using preferred parameters for the modulating signal such as using a frequency in the range 30-100 Hz, or a frequency in the range 30-60 Hz, or using a square wave for the modulating signal, or using a duty cycle of 20 to 80% for the modulating signal, or varying a frequency and/or a duty cycle of the modulating signal, or varying the frequency and/or the duty cycle of the modulating signal periodically, or using a period of the frequency and/or the duty cycle of the modulating signal which varies in the range 0.01 to 1 second, or in the range 100-150 milliseconds, as shown at 46. The method includes using preferred parameters for the carrier signal such as a frequency in the range 125 kHz-20 MHz, or a frequency in the range 11-15 Hz, or a frequency of about 13.56 MHz, as shown at 48. The method includes using a preferred field strength for the near field signal such as a field strength of 1 microTesla to 100 microTesla at a range of 4 cm or less, or a field strength of 15 microTesla at 4 cm or less, as shown at 50.

The method may further include irradiating the cells with a periodic RF signal in addition to the near field signal, as shown at 52. The method further includes using a periodic communications frequency signal for the RF signal, as shown at 54. The method further includes using a directional antenna to direct the near field signal or for controlling a radiation pattern of the near field signal, as shown at 56.

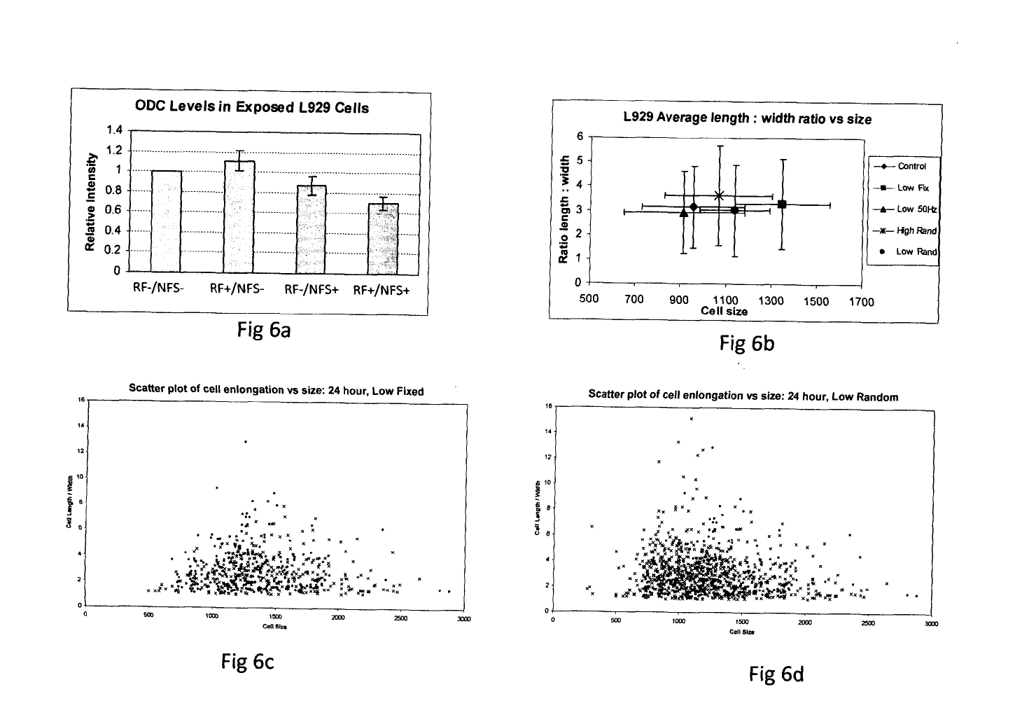

FIG. 6 shows a series of graphs illustrating experimental results. Experiments were conducted to examine changes in the level of the enzyme Ornithine Decarboxylase (ODC) as a measure for potential carcinogenic effects due to exposure to a near field signal and/or an RF signal. ODC has been established as a critical enzyme that is upregulated in the process of transformation of normal cells to cancerous cells. Cultures of murine fibroblast L929 cells (skin cells) were plated and incubated to achieve a sub-confluent culture. These cells were located close to the RF generator 3 and the near field generator 12. The experimental apparatus used was designed to minimize any thermal effects on the exposed cells, with active air circulation between and over the culture dishes and the RF generator 3 and the near field generator 12. The intensity of the fields used had sufficiently low intensity so that direct heating effects were not present.

Assays were produced to investigate the effect of the near field signal and the RF signal by growing L929 cells in a standard cell culture incubator (37° C., 5% carbon dioxide). The assays were exposed to radiation from the RF generator 3, and a pseudo-randomly modulated near field signal for 6 hours before they were assayed for ODC activity or imaged. The cells were about 10 mm above the surface of the near field generator 12 and RF generator 3. A mobile phone was used as the RF generator 3. The near field signal from the near field generator 12 comprised a 13.56 MHz carrier signal, which was modulated between 30-60 Hz with a period of about 0.1 seconds.

FIG. 6a shows the results in the absence (−) or presence (+) of the RF signal or the near field signal, indicated as RF and NFS respectively. The cells were lysed to release the cell proteins which were assayed using a monoclonal antibody that binds specifically to ODC. Total protein loading was corrected normalizing detected levels against the housekeeping protein β-actin. The assays were completed at five antibody concentrations, each in triplicate. FIG. 6a shows that under these exposure conditions there is a trend towards a reduction in ODC levels in the presence of the near field signal alone, and in combination with the RF signal.

Additional experiments were performed to determine visible changes in the cells due to the near field signal without the presence of the RF signal. The results of these additional experiments are shown in FIGS. 6b, 6c and 6d. In these experiments the cells were digitally imaged using a microscope and the cell morphology analysed by computer to determine the overall shape of each cell. This was determined as the total size, and length to width ratio of a best fit oval to each cell.

FIG. 6b shows the results of five experiments comprising “control”, “Low Fix”, “Low 50 Hz”, “High Rand”, and “Low Rand” results, showing the average cell elongation (length to width ratio) against size for several hundred imaged cells for each condition. The “control” experiment was performed without the near field signal present. The “Low Fix” experiment used a near field signal comprising an unmodulated carrier 13.56 MHz signal, and with a 3 mm gap between the near field generator and the cells. The “Low 50 Hz” experiment used a near field signal comprising the carrier 13.56 MHz signal modulated with a fixed modulating signal of 50 Hz, and with a 3 mm gap between the near field generator and the cells. The “High Rand” experiment used a near field signal comprising the carrier 13.56 MHz signal modulated between 30-60 Hz with a period of about 0.1 seconds, and with a 10 mm gap between the near field generator and the cells. The “Low Rand” experiment used a near field signal comprising the carrier 13.56 MHz signal modulated between 30-60 Hz with a period of about 0.1 seconds and with a 3 mm gap between the near field generator and the cells.

FIG. 6c shows the results of two experiments comprising “control” and “Low Fix” results. Cell elongation (length:width) and size for individual cells are shown with the “control” results plotted as dots and the “Low Fix” results plotted as crosses. The “control” experiment was performed without the near field signal present. The “Low Fix” experiment used a near field signal comprising an unmodulated carrier 13.56 MHz signal and with a 3 mm gap between the near field generator and the cells. FIG. 6c shows that the dots and crosses have a different spread which shows that there is an effect on the cells due to the presence of the near field signal.

FIG. 6d shows the results of two experiments comprising “control” and “Low Rand” results. Cell elongation (length:width) and size for individual cells are shown with the “control” results plotted as dots and the “Low Rand” results plotted as crosses. The “control” experiment was performed without the near field signal present. The “Low Rand” experiment used a near field signal comprising the carrier 13.56 MHz signal modulated between 30-60 Hz with a period of about 0.1 seconds, and with a 3 mm gap between the near field generator and the cells. FIG. 6d shows that the dots and crosses have a different spread which shows that there is an effect on the cells due to the presence of the near field signal.

FIGS. 6b, 6c and 6d show that there are visible changes on the cells due to the presence of the near field signal alone, which is a highly significant result. When this result is combined with the results shown in FIG. 6a demonstrating lower ODC levels for cells in the presence of the near field signal with or without the RF signal, the result is even more significant. This is because ODC levels are linked to cell division and are typically increased in 90% of cancers. Overall the experimental results show that consistent morphological effects were observed when L929 fibroblasts were subjected to exposure to a near field signal. The changes observed are consistent in showing not only that the fields used produce an effect, but that effect may be used to treat biological cells, for example cancerous cells.

The above embodiments are to be understood as illustrative examples of the invention. Further embodiments of the invention are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. Therapeutic apparatus for irradiating biological cells comprising a near field generator comprising an antenna and a signal generator, the near field generator being configured to generate a near field signal, wherein the near field signal substantially comprises a magnetic field, and the near field generator is configured to modulate the near field signal by a predetermined modulating signal which is adapted to vary in frequency and/or duty cycle periodically, the apparatus further comprising an RF signal generator configured to generate a periodic RF signal for irradiating the cells in addition to the near field signal.

2. (canceled)

3. Apparatus according to claim 1, wherein the near field signal comprises a carrier signal having a frequency in the range 10 kHz to 20 MHz, the carrier signal being modulated by the modulating signal having a frequency in the range 0.01 Hz to 10 kHz.

4. Apparatus according to claim 3, wherein the modulating signal has a frequency in the range 30-100 Hz.

5. Apparatus according to claim 4, wherein the modulating signal has a frequency in the range 30-60 Hz.

6. Apparatus according to claim 3, wherein the carrier signal has a frequency in the range 125 kHz-20 MHz.

7. Apparatus according to claim 6, wherein the carrier signal has a frequency in the range 11-15 MHz.

8. Apparatus according to claim 7, wherein the carrier signal has a frequency of about 13.56 MHz.

9. Apparatus according to claim 1, wherein the modulating signal is a square wave.

10-11. (canceled)

12. Apparatus according to claim 1, wherein the frequency and/or the duty cycle of the modulating signal varies with a period in the range 0.01 to 1 second.

13. Apparatus according to claim 12, wherein the frequency and/or the duty cycle of the modulating signal varies with a period in the range 100-150 milliseconds.

14. Apparatus according to claim 9, wherein the modulating signal has a duty cycle of 20 to 80%.

15. Apparatus according to claim 1, wherein the near field signal has a field strength of 1 microTesla to 100 microTesla at a range of 4 cm or less.

16. Apparatus according to claim 15, wherein the field strength is 15 microTesla at 4 cm or less.

17. (canceled)

18. Apparatus according to claim 1, wherein the RF signal is a periodic communications frequency signal.

19. Apparatus according to claim 1, wherein the antenna of the near field generator is a directional antenna.

20. Apparatus according to claim 19, comprising an antenna arrangement having a controllable radiation pattern and means for controlling the radiation pattern.

21. (canceled)

22. A method of treating biological cells comprising irradiating the cells with a near field signal using a therapeutic apparatus comprising a near field generator comprising an antenna and a signal generator, the near field generator being configured to generate the near field signal, wherein the near field signal substantially comprises a magnetic field, and the near field generator is configured to modulate the near field signal by a predetermined modulating signal which is adapted to vary in frequency and/or duty cycle periodically, the apparatus further comprising an RF signal generator configured to generate a periodic RF signal for irradiating the cells in addition to the near field signal.

23. (canceled)

24. A method according to claim 22 and further including using a near field signal comprises a carrier signal having a frequency in the range 10 kHz to 20 MHz which is modulated by the modulating signal having a frequency in the range 0.01 Hz to 10 kHz.

25. A method according to claim 24 and further including using a frequency in the range 30-100 Hz for the modulating signal.

26. A method according to claim 25 and further including using a frequency in the range 30-60 Hz for the modulating signal.

27. A method according to claim 24 and further including using a frequency in the range 125 kHz-20 MHz for the carrier signal.

28. A method according to claim 27 and further including using a frequency in the range 11-15 MHz for the carrier signal.

29. A method according to claim 28 and further including using a frequency of about 13.56 MHz for the carrier signal.

30. A method according to claim 22 and further including using a square wave for the modulating signal.

31-32. (canceled)

33. A method according to claim 22 and further including using a period of the frequency and/or the duty cycle of the modulating signal which varies in the range 0.01 to 1 second.

34. A method according to claim 33 and further including using a period of the frequency and/or the duty cycle of the modulating signal which varies in the range 100-150 milliseconds.

35. A method according to claim 22 and further including using a duty cycle of 20 to 80% for the modulating signal.

36. A method according to claim 22 and further including using a field strength of 1 microTesla to 100 microTesla at a range of 4 cm or less for the near field signal.

37. A method according to claim 36 and further including using the field strength of 15 microTesla at 4 cm or less.

38. (canceled)

39. A method according to claim 22 and further including using a periodic communications frequency signal for the RF signal.

40. A method according to claim 22 and further including using a directional antenna to direct the near field signal.

41. A method according to claim 40 and further including controlling a radiation pattern of the near field signal using the directional antenna.

42. (canceled)
